(19) United States
(12) Patent Application Publication
(10) Pub. No.: US 2024/0065960 A1
(43) Pub. Date: Feb. 29, 2024

(54) STABILIZATION OF THIOPYRIDINONE COMPOUND IN W/O COMPOSITION

(71) Applicant: L'OREAL, PARIS (FR)

(72) Inventors: Romain TACHON, Kawasaki-shi (JP); Amelie PREVOT-GUEGUINIAT, Aulnay-sous-Bois (FR); Makoto SAITO, Kawasaki-shi (JP)

(21) Appl. No.: 18/257,731

(22) PCT Filed: Dec. 10, 2021
(86) PCT No.: PCT/JP2021/046658

§ 371 Date: Jun. 15, 2023

(30) Foreign Application Priority Data
Dec. 22, 2020 (JP) 2020-212331
Jan. 19, 2021 (FR) 2100495

(51) Int. Cl. A61K 8/49; A61Q 19/02

(52) U.S. Cl. CPC A61K 8/4926; A61Q 19/02; A61K 2800/52

(57) ABSTRACT

The present invention relates to a composition comprising: (a) at least one thiopyridinone compound; (b) at least one oil; and (c) water, wherein the composition comprises a continuous oil phase comprising the (b) at least one oil, a discontinuous aqueous phase comprising the (a) thiopyridinone compound and (c) water, dispersed in the oil phase, and the weight ratio of the aqueous phase to the oil phase is 1.0 to 10.0. The present invention can provide a composition including (a) thiopyridinone compound(s) with increased stability of the (a) thiopyridinone compound(s), even when the composition is maintained for a relatively long period of time under elevated temperature.

### TECHNICAL FIELD

The present invention relates to the stabilization of a thiopyridinone compound in a composition of the W/O type.

### BACKGROUND ART

At various periods of their life, some people see the appearance on their skin, and more in particular on their hands, of darker and/or more colored spots, which give the skin heterogeneity. These spots are in particular due to a high concentration of melanin in the keratinocytes located at the surface of the skin.

The use of harmless topical depigmenting substances with good efficacy is most particularly desired for the purpose of treating pigmentation spots.

For example, arbutin, niacinamide and kojic acid are known as skin depigmenting agents.

On the other hand, WO2017/102349 discloses a new depigmenting or whitening agent, i.e., a thiopyridinone compound. The thiopyridinone compound can show strong depigmenting or whitening effects by reducing the production of melanin.

### DISCLOSURE OF INVENTION

However, it has been discovered that a thiopyridinone compound tends to be destabilized in a composition, in particular when the composition including the thiopyridinone compound is maintained for a relatively long period of time under elevated temperature.

Thus, an objective of the present invention is to provide a composition including thiopyridinone compound(s) with increased stability of the thiopyridinone compound(s), even when the composition is maintained for a relatively long period of time under elevated temperature.

The above objective can be achieved by a W/O composition comprising:
(a) at least one compound of formula (I)

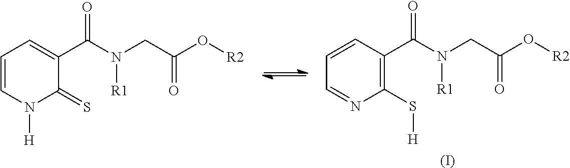

wherein

R1 denotes a radical chosen from:
a) a hydrogen atom; and
b) a saturated linear C₁-C₆ alkyl group,
   and

R2 denotes a radical chosen from:
a) a hydrogen atom;
b) a saturated linear C₁-C₁₀ alkyl group;
c) a saturated branched C₃-C₁₀ alkyl group; and
d) a C₁-C₆phenylalkyl group such as benzyl,
   or
   a salt thereof, a solvate thereof, an optical isomer thereof, or a racemate thereof;
(b) at least one oil; and
(c) water,
   wherein

the composition comprises

a continuous oil phase comprising the (b) at least one oil,

a discontinuous aqueous phase comprising the (a) at least one compound of formula (I) and (c) water, dispersed in the oil phase, and

the weight ratio of the aqueous phase to the oil phase is 1.0 to 10.0.

It is preferable that, in formula (I),

R1 denote a radical chosen from:
a) a hydrogen atom; and
b) a saturated linear C₁-C₄ alkyl radical and preferably methyl,
   and

R2 denote a radical chosen from:
a) a hydrogen atom;
b) a saturated linear C₁-C₆ alkyl group; and
c) a saturated branched C₃-C₆ alkyl group.

It is more preferable that, in formula (I),

R1 denote a radical chosen from:
a) a hydrogen atom; and
b) a methyl radical,
   and

R2 denote a radical chosen from:
a) a hydrogen atom;
b) a saturated linear C₁-C₄ alkyl group, preferably ethyl;
c) a saturated branched C₃-C₄ alkyl group, preferably isopropyl and isobutyl.

The (a) compound of formula (I) may be chosen from the following compounds:

| | Compound | |
|---|---|---|
| Structure | No. | Chemical Name |
| 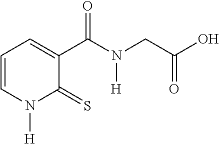 [SMILES: O=C(O)CNC(=O)c1ccc[nH]c1=S] | 1 | N-[(2-thioxo-1,2-dihydro- pyridin-3-yl)carbonyl]- glycine |
| 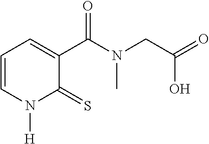 | 2 | N-methyl-N-[(2-thioxo- 1,2-dihydropyridin-3- yl)carbonyl]glycine |
| 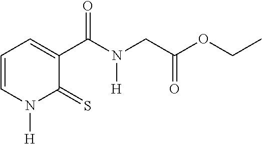 [SMILES: CCOC(=O)CNC(=O)c1ccc[nH]c1=S] | 3 | Ethyl N-[(2-thioxo-1,2- dihydropyridin-3-yl)- carbonyl]glycinate |
| 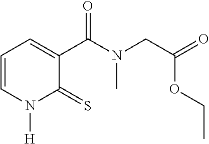 [SMILES: CCOC(=O)CN(C)C(=O)c1ccc[nH]c1=S] | 4 | Ethyl N-methyl-N-[(2- thioxo-1,2-dihydro- pyridin-3-yl)carbonyl]- glycinate |

and salts thereof, solvates thereof, optical isomers thereof, and racemates thereof.

Preferably, the (a) compound of formula (I) may be chosen from the following compounds:

| | Compound | |
|---|---|---|
| Structure | No. | Chemical Name |
| 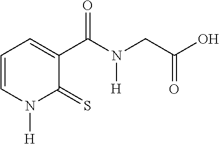 [SMILES: O=C(O)CNC(=O)c1ccc[nH]c1=S] | 1 | N-[(2-thioxo-1,2-dihydropyri- din-3-yl)carbonyl]glycine |
| 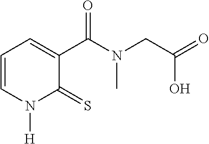 | 2 | N-methyl-N-[(2-thioxo-1,2- dihydropyridin-3-yl)carbonyl]- glycine |

and salts thereof, solvates thereof, optical isomers thereof, and racemates thereof.

The amount of the (a) compound(s) of formula (I) in the composition according to the present invention may be from 0.01% to 20% by weight, preferably from 0.05% to 10% by weight, and more preferably from 0.1% to 5% by weight, relative to the total weight of the composition.

The (b) oil may be selected from silicone oils, preferably non-volatile silicone oils, and more preferably dimethicone, phenyl trimethicone, and a mixture thereof.

The amount of the (b) oil(s) in the composition may be from 8% to 40% by weight, preferably from 9% to 35% by weight, and more preferably from 10% to 30% by weight, relative to the total weight of the composition.

The amount of the (c) water in the composition may be from 10% to 80% by weight, preferably from 20% to 75% by weight, and more preferably from 30% to 70% by weight, relative to the total weight of the composition.

The composition according to the present invention may further comprise (d) at least one lipophilic gelling agent, preferably selected from non-emulsifying silicone elastomers, and more preferably selected from dimethicone/vinyldimethicone crosspolymer, dimethicone crosspolymer and a mixture thereof.

The composition according to the present invention may further comprise (e) at least one emulsifying silicone elastomer, preferably selected from silicone elastomers comprising at least one oxyalkylenated chain and/or at least one glycerylated chain, and more preferably silicone elastomers comprising at least one oxyethylenated chain.

The composition according to the present invention may further comprise (f) at least one emulsifier different from the (e) emulsifying silicone elastomer, preferably selected from polyoxyalkylene-polysiloxane copolymers, and more preferably selected from the group consisting of PEG 10 dimethicone, PEG/PPG-18/18 dimethicone, and a mixture thereof.

The composition according to the present invention may be for whitening a keratin substance, preferably skin.

The present invention also relates to a cosmetic process, preferably a whitening process, for a keratin substance, preferably skin, comprising the step of:
applying to the keratin substance the composition according to the present invention.

Another aspect of the present invention is a use of a W/O composition comprising

a continuous oil phase comprising (b) at least one oil, and

a discontinuous aqueous phase comprising (c) water, dispersed in the oil phase, wherein the weight ratio of the aqueous phase to the oil phase is 1.0 to 10.0,

in order to stabilize (a) at least one compound of formula (I)

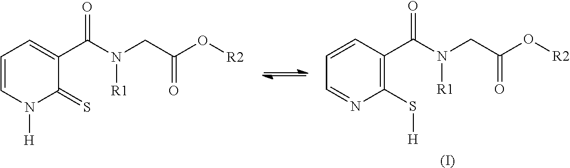

wherein

R1 denotes a radical chosen from:
a) a hydrogen atom; and
b) a saturated linear C₁-C₆ alkyl group,
   and

R2 denotes a radical chosen from:
a) a hydrogen atom;
b) a saturated linear C₁-C₁₀ alkyl group;
c) a saturated branched C₃-C₁₀ alkyl group; and
d) a C₁-C₆ phenylalkyl group such as benzyl,
   or
   a salt thereof, a solvate thereof, an optical isomer thereof, or a racemate thereof, wherein

the (a) at least one compound of formula (I) is present in the aqueous phase of the composition.

### BEST MODE FOR CARRYING OUT THE INVENTION

After diligent research, the inventors have discovered that it is possible to provide a composition including a thiopyridinone compound or thiopyridinone compounds with increased stability of the thiopyridinone compound(s), even when the composition is maintained for a relatively long period of time under elevated temperature.

Thus, the composition according to the present invention is a W/O composition and comprises:
(a) at least one compound of formula (I)

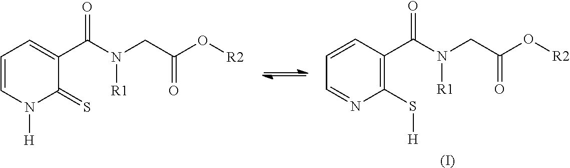

wherein

R1 denotes a radical chosen from:
a) a hydrogen atom; and
b) a saturated linear C₁-C₆ alkyl group,
   and

R2 denotes a radical chosen from:
a) a hydrogen atom;
b) a saturated linear C₁-C₁₀ alkyl group;
c) a saturated branched C₃-C₁₀ alkyl group; and
d) a C₁-C₆ phenylalkyl group such as benzyl,
   or
   a salt thereof, a solvate thereof, an optical isomer thereof, or a racemate thereof (hereafter, may be referred to as “(a) thiopyridinone compound”);
(b) at least one oil; and
(c) water,
   wherein

the composition comprises

a continuous oil phase comprising the (b) at least one oil,

a discontinuous aqueous phase comprising the (a) at least one compound of formula (I) and (c) water, dispersed in the oil phase, and

the weight ratio of the aqueous phase to the oil phase is 1.0 to 10.0.

The composition according to the present invention can show increased stability of the (a) thiopyridinone compound therein.

In other words, the composition according to the present invention can increase the stability of the (a) thiopyridinone compound therein. The term “stability” of the (a) thiopyridinone compound can be determined by the change in the amount of the (a) thiopyridinone compound in the composition according to the present invention. An increased “stability” means that the change in the amount of the (a) thiopyridinone compound is more limited.

The (a) thiopyridinone compound in a composition tends to decompose over time. Thus, the amount of the (a) thiopyridinone compound tends to reduce over time. Accordingly, an increased stability of the (a) thiopyridinone compound means that the reduction over time of the amount of the (a) thiopyridinone compound in a composition is restricted or smaller.

The composition according to the present invention can show increased stability of the (a) thiopyridinone compound therein, even when the composition is maintained for a relatively long period of time such as two months under elevated temperature such as 45° C.

The composition according to the present invention can also show increased stability of the (a) thiopyridinone compound therein, when the composition is maintained for a relatively long period of time such as two months under room temperature such as 25° C.

Therefore, the composition according to the present invention can be stored for a long period of time under both ambient and hot conditions, and in particular even under hot conditions.

In addition, the increased stability of the (a) thiopyridinone compound can provide improved or enhanced bioavailability of the (a) thiopyridinone compound which can function as a depigmenting or whitening agent. Therefore, the composition according to the present invention can provide enhanced or improved depigmenting or whitening effects.

The composition according to the present invention can also provide good texture such as less stickiness or smooth feeling to the touch.

Without being bound by theory, the stabilization of the (a) thiopyridinone compound may be attributed to, at least, the protection of the (a) thiopyridinone compound from oxidation by air by making the (a) thiopyridinone compound present in the internal aqueous phase which is surrounded by the oil phase of the composition.

Thus, the use of a W/O composition comprising
a continuous oil phase comprising (b) at least one oil, and
a discontinuous aqueous phase comprising (c) water, dispersed in the oil phase, wherein the weight ratio of the aqueous phase to the oil phase is 1.0 to 10.0
can stabilize the (a) thiopyridinone compound.

In other words, the W/O composition according to the present invention can function as a stabilizer for the (a) thiopyridinone compound, which could be an oxidization inhibitor for the (a) thiopyridinone compound.

Hereafter, the composition, use and the like according to the present invention will be described in a detailed manner.

### Composition

The composition according to the present invention comprises:
(a) at least one thiopyridinone compound;
(b) at least one oil; and
(c) water,
   wherein

the composition comprises

a continuous oil phase comprising the (b) at least one oil,

a discontinuous aqueous phase comprising the (a) thyopyridinone compound and (c) water, dispersed in the oil phase, and

the weight ratio of the aqueous phase to the oil phase is 1.0 to 10.0, preferably 1.3 to 9.0, more preferably 1.5 to 8.0, and even more preferably 1.7 to 7.0.

The (a) thiopyridinone compound, (b) oil, and (c) water, as well as the other features of the composition according to the present invention will be explained below.

### Thiopyridinone Compound

The composition according to the present invention comprises (a) at least one thiopyridinone compound. Two or more (a) thiopyridinone compounds may be used in combination. Thus, a single type of (a) thiopyridinone compound or a combination of different types of (a) thiopyridinone compounds may be used.

The (a) thiopyridinone compound may be an active ingredient or active compound in cosmetics or dermatological products. The term “active” ingredient or compound used herein means an ingredient or compound which has a cosmetic or dermatological active property, such as anti-oxidant, whitening, UV-filtering effects and anti-bacterial effects. The (a) thiopyridinone compound used in the present invention can function as a depigmenting, bleaching or whitening agent, and thus the composition according to the present invention may be used as a whitening product or as a cosmetic composition for a whitening keratin substance.

The (a) thiopyridinone compound may be used as an agent for depigmenting, bleaching or whitening the skin, body hairs, the eyelashes or head hair, and also the lips and/or the nails, and preferably the skin, in particular for eliminating pigmentation spots or senescence spots, and/or as an anti-tanning agent.

The (a) thiopyridinone compound is represented by the following formula (I)

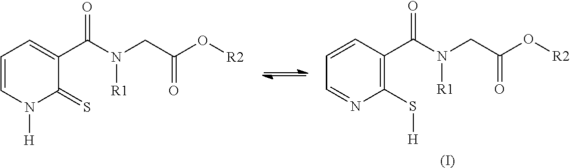

wherein

R1 denotes a radical chosen from:
a) a hydrogen atom; and
b) a saturated linear C₁-C₆ alkyl group,
   and

R2 denotes a radical chosen from:
a) a hydrogen atom;
b) a saturated linear C₁-C₁₀ alkyl group;
c) a saturated branched C₃-C₁₀ alkyl group; and
d) a C₁-C₆ phenylalkyl group such as benzyl,
   or
   a salt thereof, a solvate thereof, an optical isomer thereof, or a racemate thereof.

The salts of the compounds of formula (I) comprise the conventional non-toxic salts of said compounds, such as those formed from an acid or base.

As salts of the compounds of formula (I), mention may be made of:
the salts obtained by addition of the compound of formula (I) (when it comprises an acid group)
to a mineral base, such as sodium hydroxide, potassium hydroxide, calcium hydroxide, ammonium hydroxide, magnesium hydroxide, lithium hydroxide, and sodium, potassium or calcium carbonate or hydrogen carbonate for example;
   or
to an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made in particular of 2-amino-2-methylpropanol, ethanolamine, triethanolamine, 2-dimethylaminopropanol, 2-amino-2-(hydroxymethyl)-1,3-propanediol and 3-(dimethylamino)propylamine.

Mention may also be made of the salts of amino acids, for instance lysine, arginine, guanidine, glutamic acid and aspartic acid. Advantageously, the salts of the compounds of formula (I) (when it comprises an acid group) may be chosen from alkali metal or alkaline-earth metal salts such as sodium, potassium, calcium or magnesium salts; ammonium salts.

The acceptable solvates of the compounds described in the present invention comprise conventional solvates such as those formed during the preparation of said compounds owing to the presence of solvents. Mention may be made, by way of example, of the solvates due to the presence of water or of linear or branched alcohols, such as ethanol or isopropanol. The optical isomers are, in particular, enantiomers and diastereoisomers.

Preferentially, the linear or branched groups may be chosen from methyl, ethyl, propyl, isopropyl, butyl, isobutyl and tert-butyl, pentyl, hexyl, heptyl, octyl, nonyl and decyl.

More preferentially, the saturated linear or branched alkyl groups may be chosen from methyl, ethyl, propyl, isopropyl, butyl, isobutyl and tert-butyl, pentyl, hexyl, heptyl and octyl.

The compound a) is disclosed in the PubCHEM database (No. 47329290) http://pubchem.ncbi.nlm.nih.gov/compound/47329290?from=summary#section=Top entry:2010 Nov. 26.

The compound b) CAS>1240664-41-8 is described in the publication:

Synthesis of N-(2-mercaptopyridyl-3-formyl)-N-alkyl glycine and the corresponding disulfides

Luo, Y. L.; Yang, Z. X.; Peng, S. X.

Div. Med. Chem., China Pharm. Univ., Nanjing, 210009, Peop. Rep. China

Yaoxue Xuebao (1990), 25(5), 374-8.

Preferably, the (a) thiopyridinone compounds of formula (I) have the following meanings:

R1 denotes a radical chosen from
a) a hydrogen atom; and
b) a saturated linear C₁-C₄ alkyl radical and preferably methyl,
   and

R2 denotes a radical chosen from:
a) a hydrogen atom;
b) a saturated linear C₁-C₆ alkyl group; and
c) a saturated branched C₃-C₆ alkyl group.

More preferably, the (a) thiopyridinone compounds of formula (I) have the following meanings:

R1 denotes a radical chosen from:
a) a hydrogen atom; and
b) a methyl radical,
   and

R2 denotes a radical chosen from:
a) a hydrogen atom;
b) a saturated linear C₁-C₄ alkyl group, preferably ethyl;
c) a saturated branched C₃-C₄ alkyl group, preferably isopropyl and isobutyl.

The (a) thiopyridinone compound of formula (I) may be chosen from the following compounds:

| | Compound | |
|---|---|---|
| Structure | No. | Chemical Name |
| 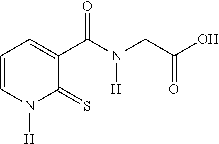 [SMILES: O=C(O)CNC(=O)c1ccc[nH]c1=S] | 1 | N-[(2-thioxo-1,2-dihydro- pyridin-3-yl)carbonyl]- glycine |
| 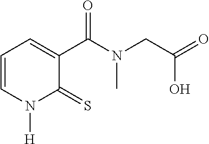 | 2 | N-methyl-N-[(2-thioxo- 1,2-dihydropyridin-3-yl)- carbonyl]glycine |
| 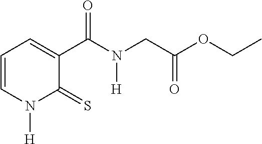 [SMILES: CCOC(=O)CNC(=O)c1ccc[nH]c1=S] | 3 | Ethyl N-[(2-thioxo-1,2- dihydropyridin-3-yl)- carbonyl]glycinate |
| 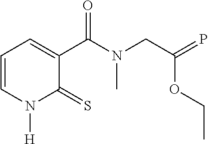 | 4 | Ethyl N-methyl-N-[(2- thioxo-1,2-dihydro- pyridin-3-yl)carbonyl]- glycinate |

and salts thereof, solvates thereof, optical isomers thereof, and racemates thereof.

Preferably, the (a) thiopyridinone compound of formula (I) may be chosen from the following compounds:

| | Compound | |
|---|---|---|
| Structure | No. | Chemical Name |
| 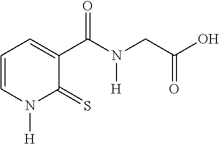 [SMILES: O=C(O)CNC(=O)c1ccc[nH]c1=S] | 1 | N-[(2-thioxo-1,2- dihydropyridin-3-yl)- carbonyl]glycine |
| 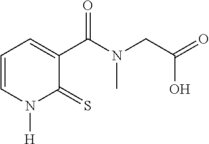 | 2 | N-methyl-N-[(2- thioxo-1,2-dihydro- pyridin-3-yl)- carbonyl]glycine |

and salts thereof, solvates thereof, optical isomers thereof, and racemates thereof.

More preferably, the (a) thiopyridinone compound may be N-[(2-thioxo-1,2-dihydropyridin-3-yl)carbonyl]glycine.

The (a) thiopyridinone compound can be prepared in accordance with the process described in, for example, WO 2017/102349, which is herein incorporated by reference.

The amount of the (a) thiopyridinone compound(s) in the composition according to the present invention may be 0.01% by weight or more, preferably 0.05% by weight or more, and more preferably 0.1% by weight or more, relative to the total weight of the composition. It may be even more preferable that the amount of the (a) thiopyridinone compound(s) in the composition according to the present invention be 0.3% by weight or more, relative to the total weight of the composition.

On the other hand, the amount of the (a) thiopyridinone compound(s) in the composition according to the present invention may be 20% by weight or less, preferably 10% by weight or less, and more preferably 5% by weight or less, relative to the total weight of the composition. It may be even more preferable that the amount of the (a) thiopyridinone compound(s) in the composition according to the present invention be 3% by weight or less, relative to the total weight of the composition.

The amount of the (a) thiopyridinone compound(s) in the composition according to the present invention may range from 0.01% to 20% by weight, preferably from 0.05% to 10% by weight, more preferably from 0.1% to 5% by weight, relative to the total weight of the composition. It may be even more preferable that the amount of the (a) thiopyridinone compound(s) in the composition according to the present invention be from 0.3% to 3% by weight, relative to the total weight of the composition.

### Oil

The composition according to the present invention comprises (b) at least one oil. If two or more (b) oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (b) oil may be a non-polar oil such as a hydrocarbon oil, a silicone oil, or the like; a polar oil such as a plant or animal oil and an ester oil or an ether oil; or a mixture thereof.

The (b) oil may be selected from the group consisting of oils of plant or animal origin, synthetic oils, silicone oils, hydrocarbon oils, and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, etc.; cyclic organopolysiloxanes such as cyclopentasiloxane, cyclohexasiloxane, octamethylcyclotetrasiloxane, decamethylcyclopentasiloxane, dodecamethylcyclohexasiloxane, etc.; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

If they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of a type such as dimethylsiloxane/methylalkylsiloxane, such as Silicone Volatile® FZ 3109 sold by the company Union Carbide, of the formula:

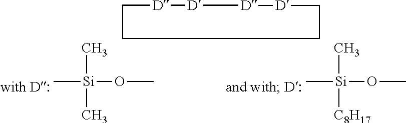

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 1976, pp. 27-32, Todd & Byers, Volatile Silicone Fluids for Cosmetics. The viscosity of the silicones is measured at 25° C. according to ASTM standard, 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

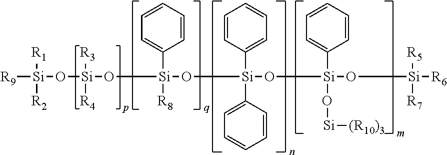

in which

R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and

m, n, p, and q are, independently of each other, integers of 0 to 900 inclusive, preferably 0 to 500 inclusive, and more preferably 0 to 100 inclusive,

with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, etc.; hydrogenated polyisobutene, isoeicosan, and decene/butene copolymer; and mixtures thereof.

The term “fatty” in the fatty alcohol means the inclusion of a relatively large number of carbon atoms. Thus, alcohols, which have 4 or more, preferably 6 or more, and more preferably 12 or more carbon atoms, are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. It is more preferable that any linear or branched, saturated C₁₆-C₂₀ fatty alcohols be used. It is even more preferable that branched C₁₆-C₂₀ fatty alcohols be used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from cetyl alcohol, octyldodecanol, hexyldecanol, and mixtures thereof.

The (b) oil may be chosen from volatile oils, non-volatile oils, and mixtures thereof.

The (b) oil may be chosen from polar oils, non-polar oils, and mixtures thereof.

The (b) oil may be selected from plant oils, silicone oils, and mixtures thereof.

It is preferable that the (b) oil be selected from silicone oils, more preferably non-volatile silicone oils, and even more preferably dimethicone, phenyl trimethicone, and a mixture thereof.

The amount of the (b) oil(s) in the composition according to the present invention may be 8% by weight or more, preferably 9% by weight or more, and more preferably 10% by weight or more, relative to the total weight of the composition.

The amount of the (b) oil(s) in the composition according to the present invention may be 40% by weight or less, preferably 35% by weight or less, and more preferably 30% by weight or less, relative to the total weight of the composition.

The amount of the (b) oil(s) in the composition according to the present invention may be from 8% to 40% by weight, preferably from 9% to 35% by weight, and more preferably from 10% to 30% by weight, relative to the total weight of the composition.

### Oil Phase

The composition according to the present invention comprises a continuous oil phase. The continuous oil phase comprises the (b) oil(s). The continuous oil phase may further comprise any lipophilic ingredient with the (b) oil(s).

The amount of the continuous oil phase in the composition according to the present invention may be 8% by weight or more, preferably 9% by weight or more, and more preferably 10% by weight or more, relative to the total weight of the composition.

The amount of the continuous oil phase in the composition according to the present invention may be 50% by weight or less, preferably 40% by weight or less, and more preferably 30% by weight or less, relative to the total weight of the composition.

The amount of the continuous oil phase in the composition according to the present invention may be from 8% to 50% by weight, preferably from 9% to 40% by weight, and more preferably from 10% to 30% by weight, relative to the total weight of the composition.

### Water

The composition according to the present invention comprises (c) water.

The amount of the (c) water in the composition according to the present invention may be 10% by weight or more, preferably 20% by weight or more, and more preferably 30% by weight or more, relative to the total weight of the composition. It may be even more preferable that the amount of the (c) water in the composition according to the present invention be 40% by weight or more, relative to the total weight of the composition.

On the other hand, the amount of the (c) water in the composition according to the present invention may be 80% by weight or less, preferably 75% by weight or less, and more preferably 70% by weight or less, relative to the total weight of the composition. It may be even more preferable that the amount of the (c) water in the composition according to the present invention be 65% by weight or less, relative to the total weight of the composition.

The amount of the (c) water in the composition according to the present invention may be from 10% to 80% by weight, preferably from 20% to 75% by weight, and more preferably from 30% to 70% by weight, relative to the total weight of the composition. It may be even more preferable that the amount of the (c) water in the composition according to the present invention be from 40% to 65% by weight, relative to the total weight of the composition.

### Aqueous Phase

The composition according to the present invention comprises a discontinuous aqueous phase. The discontinuous aqueous phase is dispersed in the continuous phase in the composition.

The composition according to the present invention can comprise a plurality of discontinuous aqueous phases which can be dispersed in the continuous oil phase in the composition.

The discontinuous aqueous phase comprises the (a) compound(s) of formula (I) explained above and the (c) water. The discontinuous aqueous phase may further comprise any hydrophilic ingredient with the (c) water.

The amount of the discontinuous aqueous phase in the composition according to the present invention may be 10% by weight or more, preferably 20% by weight or more, and more preferably 30% by weight or more, relative to the total weight of the composition.

The amount of the discontinuous aqueous phase in the composition according to the present invention may be 85% by weight or less. It may be preferable that the amount of the discontinuous aqueous phase in the composition according to the present invention be 80% by weight or less, more preferably 75% by weight or less, and even more preferably 70% by weight or less, relative to the total weight of the composition.

The amount of the discontinuous aqueous phase in the composition according to the present invention may be from 10% to 85% by weight, preferably from 20% to 85% by weight, and more preferably from 30% to 85% by weight, relative to the total weight of the composition.

### Weight Ratio of Aqueous Phase/Oil Phase

According to the present invention, the weight ratio of the (discontinuous) aqueous phase to the (continuous) oil phase is 1.0 to 10.0, preferably 1.3 to 9.0, more preferably 1.5 to 8.0, and even more preferably 1.7 to 7.0.

In one embodiment, the weight ratio of the (discontinuous) aqueous phase to the (continuous) oil phase may be 1.0 or more, preferably 1.3 or more, more preferably 1.5 or more, and even more preferably 1.7 or more.

In another embodiment, the weight ratio of the (discontinuous) aqueous phase to the (continuous) oil phase may be 10.0 or less, preferably 9.0 or less, more preferably 8.0 or less, and even more preferably 7.0 or less.

### Lipophilic Gelling Agent

The composition according to the present invention may comprise (d) at least one lipophilic gelling agent. If two or more lipophilic gelling agents are used, they may be the same or different.

For the purposes of the present invention, the term “lipophilic gelling agent” means a compound that is capable of gelling the oily phase of the compositions according to the present invention.

The gelling agent is lipophilic and is thus present in the oily phase of the composition.

The gelling agent is liposoluble or lipodispersible.

It is preferable that the (d) lipophilic gelling agent be selected from silicone elastomers.

The term “silicone elastomer” or “organopolysiloxane elastomer” means a supple, deformable organopolysiloxane with viscoelastic properties and especially with the consistency of a sponge or a supple sphere. Its modulus of elasticity is such that this material withstands deformation and has a limited ability to extend and to contract. This material is capable of regaining its original shape after stretching. It is more particularly a crosslinked organopolysiloxane elastomer.

Thus, the organopolysiloxane elastomer can be obtained by a crosslinking addition reaction of diorganopolysiloxane containing at least one hydrogen bonded to silicon and of diorganopolysiloxane having ethylenically unsaturated groups bonded to silicon, in particular in the presence of a platinum catalyst; or by a dehydrogenation crosslinking condensation reaction between a diorganopolysiloxane comprising hydroxyl end groups and a diorganopolysiloxane containing at least one hydrogen bonded to silicon, in particular in the presence of an organotin compound; or by a crosslinking condensation reaction of a diorganopolysiloxane comprising hydroxyl end groups and of a hydrolysable organopolysilane; or by thermal crosslinking of organopolysiloxane, in particular in the presence of an organic peroxide catalyst; or by crosslinking of organopolysiloxane via high-energy radiation, such as gamma rays, ultraviolet rays or an electron beam.

Preferably, the organopolysiloxane elastomer is obtained by a crosslinking addition reaction (A) of diorganopolysiloxane containing at least two hydrogens each bonded to a silicon, and (B) of diorganopolysiloxane containing at least two ethylenically unsaturated groups bonded to silicon, especially in the presence of (C) a platinum catalyst, as described, for instance, in patent application EP-A-295 886.

In particular, the organopolysiloxane elastomer may be obtained by reaction of dimethylpolysiloxane bearing dimethylvinylsiloxy end groups and of methylhydrogenopolysiloxane bearing trimethylsiloxy end groups, in the presence of a platinum catalyst.

Compound (A) is the base reactant for the formation of elastomeric organopolysiloxane, and the crosslinking takes place via an addition reaction of compound (A) with compound (B) in the presence of the catalyst (C).

Compound (A) is in particular an organopolysiloxane containing at least two hydrogen atoms bonded to different silicon atoms in each molecule.

Compound (A) may have any molecular structure, especially a linear-chain or branched-chain structure or a cyclic structure.

Compound (A) may have a viscosity at 25° C. ranging from 1 to 50,000 centistokes, especially so as to be readily miscible with compound (B).

The organic groups bonded to the silicon atoms of compound (A) may be alkyl groups such as methyl, ethyl, propyl, butyl, octyl; substituted alkyl groups such as 2-phenylethyl, 2-phenylpropyl or 3,3,3-trifluoropropyl; aryl groups such as phenyl, tolyl, xylyl; substituted aryl groups such as phenylethyl; and substituted monovalent hydrocarbon-based groups such as an epoxy group, a carboxylate ester group or a mercapto group.

Compound (A) may thus be chosen from trimethylsiloxy-terminated methylhydrogenopolysiloxanes, trimethylsiloxy-terminated dimethylsiloxanelmethylhydrogenosiloxane copolymers, and dimethylsiloxanelmethylhydrogenosiloxane cyclic copolymers.

Compound (B) is advantageously a diorganopolysiloxane containing at least two lower alkenyl groups (for example C₂-C₄); the lower alkenyl group may be chosen from vinyl, allyl and propenyl groups. These lower alkenyl groups may be located in any position on the organopolysiloxane molecule, but are preferably located at the ends of the organopolysiloxane molecule. The organopolysiloxane (B) may have a branched-chain, linear-chain, cyclic or network structure, but the linear-chain structure is preferred. Compound (B) may have a viscosity ranging from the liquid state to the gum state. Preferably, compound (B) has a viscosity of at least 100 centistokes at 25° C.

Besides the above-mentioned alkenyl groups, the other organic groups bonded to the silicon atoms in compound (B) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The organopolysiloxanes (B) can be chosen from methylvinylpolysiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethylsiloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxanes comprising dimethylvinylsiloxy end groups, and dimethylsiloxane-methyl(3,3,3-trifluoropropyl)siloxane copolymers comprising dimethylvinylsiloxy end groups.

In particular, the organopolysiloxane elastomer can be obtained by reaction of dimethylpolysiloxane comprising dimethylvinylsiloxy end groups and of methylhydropolysiloxane comprising trimethylsiloxy end groups, in the presence of a platinum catalyst.

Advantageously, the sum of the number of ethylenic groups per molecule in compound (B) and of the number of hydrogen atoms bonded to silicon atoms per molecule in compound (A) is at least 5.

It is advantageous for compound (A) to be added in an amount such that the molecular ratio between the total amount of hydrogen atoms bonded to silicon atoms in compound (A) and the total amount of all the ethylenically unsaturated groups in compound (B) is within the range from 1.5/1 to 20/1.

Compound (C) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

Catalyst (C) is preferably added in an amount of from 0.1 to 1,000 parts by weight and better still from 1 to 100 parts by weight, as clean platinum metal, per 1,000 parts by weight of the total amount of compounds (A) and (B).

It is preferable that the elastomer be a non-emulsifying silicone or organopolysiloxane elastomer.

The term “non-emulsifying” defines organopolysiloxane elastomers not containing a hydrophilic chain and in particular not containing polyoxyalkylene units (especially polyoxyethylene or polyoxypropylene units) or a polyglyceryl unit. Thus, according to a specific form of the present invention, the composition comprises an organopolysiloxane elastomer devoid of polyoxyalkylene units and of polyglyceryl unit.

In particular, the silicone elastomer used in the present invention is chosen from Dimethicone Crosspolymer (INCI name), Vinyl Dimethicone Crosspolymer (INCI name), Dimethicone/Vinyl Dimethicone Crosspolymer (INCI name) or Dimethicone Crosspolymer-3 (INCI name).

The organopolysiloxane elastomer particles may be conveyed in the form of a gel formed from an elastomeric organopolysiloxane included in at least one hydrocarbon-based oil and/or one silicone oil. In these gels, the organopolysiloxane particles are often non-spherical particles.

Non-emulsifying elastomers are especially described in patents EP 242 219, EP 285 886 and EP 765 656 and in patent application JP-A-61-194 009.

The silicone elastomer is generally provided in the form of a gel, a paste or a powder but advantageously in the form of a gel in which the silicone elastomer is dispersed in a linear silicone oil (dimethicone) or cyclic silicone oil (e.g.: cyclopentasiloxane), advantageously in a linear silicone oil.

Non-emulsifying elastomers that may more particularly be used include those sold under the names KSG-6, KSG-15, KSG-16, KSG-18, KSG-41, KSG-42, KSG-43 and KSG-44 by the company Shin-Etsu, DC9040 and DC9041 by Dow Corning and SFE 839 by the company General Electric.

According to a particular mode, use is made of a gel of silicone elastomer dispersed in a silicone oil chosen from a non-exhaustive list comprising cyclopentadimethylsiloxane, dimethicones, dimethylsiloxanes, methyl trimethicone, phenyl methicone, phenyl dimethicone, phenyl trimethicone and cyclomethicone, preferably a linear silicone oil chosen from polydimethylsiloxanes (PDMS) or dimethicones with a viscosity at 25° C. ranging from 1 to 500 cSt, optionally modified with optionally fluorinated aliphatic groups, or with functional groups such as hydroxyl, thiol and/or amine groups.

Mention may be made especially of the compounds having the following INCI names:
dimethicone/vinyl dimethicone crosspolymer, such as USG-105 and USG-107A from the company Shin-Etsu; DC9506 and DC9701 from the company Dow Corning;
dimethicone/vinyl dimethicone crosspolymer (and) dimethicone, such as KSG-6 and KSG-16 from the company Shin-Etsu;
dimethicone/vinyl dimethicone crosspolymer (and) cyclopentasiloxane, such as KSG-15; cyclopentasiloxane (and) dimethicone crosspolymer, such as DC9040, DC9045 and DC5930 from the company Dow Corning;
dimethicone (and) dimethicone crosspolymer, such as DC9041 from the company Dow Corning.
dimethicone (and) dimethicone crosspolymer, such as Dow Corning EL-9240 Silicone Elastomer Blend from the company Dow Corning (mixture of polydimethylsiloxane crosslinked with hexadiene/polydimethylsiloxane (2 cSt));
C₄-₂₄ alkyl dimethicone/divinyl dimethicone crosspolymer, such as NuLastic Silk MA from the company Alzo.

Mention may in particular be made, as examples of silicone elastomers dispersed in a linear silicone oil which can advantageously be used according to the present invention, of the following references:
dimethicone/vinyl dimethicone crosspolymer (and) dimethicone, such as KSG-6 and KSG-16 from the company Shin-Etsu;
dimethicone (and) dimethicone crosspolymer, such as DC9041, Dow Corning EL-9240 Silicone Elastomer Blend from the company Dow Corning.

The organopolysiloxane elastomer particles may also be used in powder form: mention may be made especially of the powders sold under the names Dow Corning 9505 Powder and Dow Corning 9506 Powder by the company Dow Corning, these powders having the INCI name: dimethicone/vinyl dimethicone crosspolymer.

The organopolysiloxane powder may also be coated with silsesquioxane resin, as described, for example, in U.S. Pat. No. 5,538,793. Such elastomeric powders are sold under the names KSP-100, KSP-101, KSP-102, KSP-103, KSP-104 and KSP-105 by the company Shin-Etsu, and have the INCI name: vinyl dimethicone/methicone silsesquioxane crosspolymer.

As examples of organopolysiloxane powders coated with silsesquioxane resin that may advantageously be used according to the present invention, mention may be made especially of the reference KSP-100 from the company Shin-Etsu.

As preferred lipophilic gelling agent of organopolysiloxane elastomer type, mention may be made especially of crosslinked organopolysiloxane elastomers chosen from Dimethicone Crosspolymer (INCI name), Dimethicone (and) Dimethicone Crosspolymer (INCI name), Vinyl Dimethicone Crosspolymer (INCI name), DimethiconeNinyl Dimethicone Crosspolymer (INCI name), Dimethicone Crosspolymer-3 (INCI name), and in particular Dimethicone Crosspolymer (INCI name)

It is preferable that the (d) lipophilic gelling agent be selected from non-emulsifying silicone elastomers, and more preferably selected from dimethicone/vinyldimethicone crosspolymer, dimethicone crosspolymer and a mixture thereof.

The amount of the (d) lipophilic gelling agent(s) in the composition according to the present invention may be 20% by weight or less, preferably 15% by weight or less, and more preferably 10% by weight or less, relative to the total weight of the composition.

The amount of the (d) lipophilic gelling agent(s) in the composition according to the present invention may be 0.1% by weight or more, preferably 0.5% by weight or more, and more preferably from 1% by weight or more, relative to the total weight of the composition.

Thus, the amount of the (d) lipophilic gelling agent(s) in the composition according to the present invention may range from 0.1% to 20% by weight, preferably from 0.5% to 15% by weight, and more preferably from 1% to 10% by weight, relative to the total weight of the composition.

### Emulsifying Silicone Elastomer

The composition according to the present invention includes (e) at least one emulsifying silicone elastomer. If two or more emulsifying silicone elastomers are used, they may be the same or different.

The term “silicone elastomer” is intended to mean a partially or completely crosslinked organopolysiloxane, which is a flexible and deformable material having viscoelastic properties. Its modulus of elasticity is such that this material withstands deformation and has a limited capacity for extension and contraction. This material is capable of returning to its original shape following stretching.

The term “emulsifying” of “emulsifying silicone elastomer” means that the silicone elastomer is capable of emulsifying, or has a function as an emulsifier.

The (e) emulsifying silicone elastomer is generally introduced into the fatty phase of the composition according to the present invention, and can be a part of this fatty phase.

The (e) emulsifying silicone elastomer used according to the present invention may be a crosslinked elastomeric organopolysiloxane comprising at least one hydrophilic chain, it being possible for this chain to be in particular oxyalkylenated or glycerylated. The (e) emulsifying silicone elastomer can therefore be chosen from silicone elastomers comprising at least one oxyalkylenated chain and/or at least one glycerylated chain.

It is preferable that the (e) emulsifying silicone elastomer comprise at least one crosslinked silicone polymer with at least one hydrophilic moiety. The hydrophilic moiety may comprise at least one polyoxyalkylene chain and/or at least one polyglyceryl chain.

The silicone elastomer comprising at least one oxyalkylenated chain can be obtained in particular by addition reaction and crosslinking of a diorganopolysiloxane containing at least two hydrogens each linked to a silicon (A1), and of a polyoxyalkylene having at least two ethylenically unsaturated groups (B1), in particular in the presence of a catalyst (C1), in particular a platinum catalyst, as described, for example, in documents U.S. Pat. Nos. 5,236,986 and 5,412,004.

Compound (A1) is the base compound for the formation of elastomeric organopolysiloxane, and the crosslinking takes place via an addition reaction of compound (A1) with compound (B1) in the presence of catalyst (C1).

Compound (B1) is advantageously an oxyethylenated and/or oxypropylenated compound containing at least two vinyl groups in the α-ω position of the silicone chain, which will react with Si—H bonds of compound (A1). Compound (B1) may in particular be a polyoxyalkylene (in particular polyoxyethylene and/or polyoxypropylene) containing dimethylvinylsiloxy end groups.

The organic groups linked to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A1) may thus be chosen from methylhydrogenopolysiloxanes containing trimethylsiloxy end groups, dimethylsiloxane-methylhydrogenosiloxane copolymers containing trimethylsiloxy end groups, cyclic dimethylsiloxane-methylhydrogenosiloxane copolymers and di-methylsiloxane-methylhydrogenosiloxanelaurylmethyl-siloxane copolymers containing trimethylsiloxy end groups.

Compound (C1) is the crosslinking reaction catalyst, and is in particular chosen from chloroplatinic acid, chloroplatinic acid-olefin complexes, chloroplatinic acid-alkenylsiloxane complexes, chloroplatinic acid-diketone complexes, platinum black and platinum on a support.

The catalyst (C1) is preferably added in an amount of from 0.1 to 1,000 parts by weight, better still from 1 to 100 parts by weight, as clean platinum metal, per 1,000 parts by weight of the total amount of compounds (A1) and (B1).

In particular, the silicone elastomer comprising at least one oxyalkylenated chain can be obtained by reaction of polyoxyalkylene (in particular polyoxyethylene and/or polyoxypropylene) containing dimethylvinylsiloxy end groups and of methylhydrogenopolysiloxane containing trimethylsiloxy end groups, in the presence of platinum catalyst.

The silicone elastomer comprising at least one oxyalkylenated chain, used according to the present invention, is preferably a silicone elastomer comprising at least one oxyethylenated chain.

In addition, the silicone elastomer comprising at least one oxyalkylenated chain is preferably carried in the form of a gel in at least one hydrocarbon-based oil and/or one silicone oil. Thus, the (e) emulsifying silicone elastomer can be in the form of a gel. In these gels, the elastomer comprising at least one oxyalkylenated chain is commonly in the form of non-spherical particles.

Polyoxyalkylenated silicone elastomers were in particular described in documents U.S. Pat. Nos. 5,236,986, 5,412,004, 5,837,793 and 5,811,487, the content of which is incorporated by way of reference.

As silicone elastomers comprising at least one oxyethylenated chain, use may in particular be made of those sold by the company Shin Etsu under the names of
Dimethicone/PEG-10 Dimethicone vinyl dimethicone crosspolymer, such as that sold under the name of KS G-21 (at 27% in terms of active material),
PEG-10 Dimethicone Crosspolymer, such as that sold under the name of KSG-20 (at 100% in terms of active material,
Dimethicone/PEG-10/15 crosspolymer, such as that sold under the name of KSG-210 (at 25% in terms of active material of silicone elastomer in silicone oil),
Cyclopentasiloxane (and) Dimethicone/PEG-10/15 crosspolymer, such as that sold under the name of KSG-240,
Mineral oil (and) PEG-15/lauryl dimethicone crosspolymer, such as that sold under the name of KSG-310,
Isododecane (and) PEG-15/lauryl dimethicone crosspolymer, such as that sold under the name of KSG-320,
Isododecane (and) PEG-15/lauryl polydimethylsiloxyethyl dimethicone crosspolymer, such as that sold under the name of KSG-320Z,
Cyclopentasiloxane (and) PEG-15/lauryl polydimethylsiloxyethyl dimethicone crosspolymer, such as that sold under the name of KSG-350Z,
Dimethicone (and) PEG-15/lauryl polydimethylsiloxyethyl dimethicone crosspolymer, such as those sold under the names of KSG-360Z, and KSG-380Z (25-45% in terms of active material of silicone elastomer in silicone oil),
   or

those sold by the company Dow Corning under the names of
PEG-12 dimethicone crosspolymer, such as that sold under the name of DC9010 (at 11% in terms of active material), and
Cyclopentasiloxane (and) PEG-12 Dimethicone crosspolymer, such as that sold under the name of DC9011 (at 91% in terms of active material).

These products are generally in the form of oily gels containing the particles of silicone elastomer.

Use is preferably made of Dimethicone/PEG-10/15 crosspolymer, such as that sold under the name of KSG-210, PEG/15 lauryl dimethicone crosspolymer, such as that sold under the name of KSG-320, and PEG-15/lauryl polydimethylsiloxyethyl dimethicone crosspolymer, such as that sold under the name of KSG-380Z.

The (a) emulsifying silicone elastomer may also be chosen from silicone elastomers comprising at least one glycerylated chain.

The silicone elastomer comprising at least one glycerylated chain can be obtained in particular by addition reaction and crosslinking of a diorganopolysiloxane containing at least one hydrogen linked to the silicon (A2) and of a polyglycerylated compound having ethylenically unsaturated groups (B2), in particular in the presence of a catalyst (C2), in particular a platinum catalyst.

In particular, the organopolysiloxane may be obtained by reaction of a polyglycerylated compound containing dimethylvinylsiloxy end groups and of methylhydrogenopolysiloxane containing trimethylsiloxy end groups, in the presence of a platinum catalyst.

Compound (A2) is the base compound for the formation of elastomeric organopolysiloxane, and the crosslinking takes place via an addition reaction of compound (A) with compound (B2) in the presence of catalyst (C2).

Compound (A2) is in particular an organopolysiloxane having at least two hydrogen atoms linked to distinct silicon atoms in each molecule. Compound (A2) may have a viscosity at 25° C., ranging from 1 to 50 000 centistokes, in particular so as to be readily miscible with compound (B2).

The organic groups linked to the silicon atoms of compound (A2) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from methyl, phenyl and lauryl groups.

Compound (A2) may thus be chosen from methylhydrogenopolysiloxanes containing trimethylsiloxy end groups, dimethylsiloxane-methylhydrogenosiloxane copolymers containing trimethylsiloxy end groups, cyclic dimethylsiloxane-methylhydrogenosiloxane copolymers, and dimethylsiloxane-methylhydrogenosiloxane-laurylmethyl-siloxane copolymers containing trimethylsiloxy end groups.

Compound (B2) may be a polyglycerylated compound corresponding to formula (B′2) below:

CₘH₂ₘ₋₁—O-[Gly]ₙ-CₘH₂ₘ₋₁ (B′2)

in which m is an integer ranging from 2 to 6, n is an integer ranging from 2 to 200, preferably from 2 to 100, preferentially from 2 to 50, better still from 2 to 20, even better still from 2 to 10, and even better still from 2 to 5, and in particular n is equal to 3; and Gly denotes:

CH₂—CH(OH)—CH₂—O— or —CH₂—CH(CH₂OH)—O—

Advantageously, the sum of the number of ethylenic groups per molecule of compound (B2) and of the number of hydrogen atoms linked to silicon atoms per molecule of compound (A2) is at least 4.

It is advantageous for compound (A2) to be added in an amount such that the molecular ratio of the total amount of hydrogen atoms linked to silicon atoms in compound (A2) to the total amount of all the ethylenically unsaturated groups in compound (B2) is within the range of from 1/1 to 20/1.

Compound (C2) is the crosslinking reaction catalyst, and is in particular chosen from chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in an amount of from 0.1 to 1,000 parts by weight, better still from 1 to 100 parts by weight, as clean platinum metal, per 1,000 parts by weight of the total amount of compounds (A2) and (B2).

The silicone elastomer comprising at least one glycerylated chain, used according to the present invention, is generally in the form of a gel as a mixture with at least one hydrocarbon-based oil and/or one silicone oil. In these gels, the elastomer comprising at least one glycerylated chain is commonly in the form of non-spherical particles.

Such elastomers are in particular described in document WO-A-2004/024798.

As the silicone elastomer comprising at least one glycerylated chain, use may be made of those sold by the company Shin Etsu under the names of
Dimethicone/Polyglycerin-3 Crosspolymer, such as that sold under the name of KSG-710 (containing 25% active material),
Mineral oil (and) Lauryl dimethicone/polyglycerin-3 crosspolymer, such as that sold under the name of KSG-810,
Isododecane (and) Lauryl dimethicone/polyglycerin-3 crosspolymer, such as that sold under the name of KSG-820,
Triethylhexanoin (and) Lauryl dimethicone/polyglycerin-3 crosspolymer, such as that sold under the name of KSG-830, and
Squalane (and) Lauryl dimethicone/polyglycerin-3 crosspolymer, such as that sold under the name of KSG-840.

It is preferable that the (e) emulsifying silicone elastomer be selected from silicone elastomers comprising at least one oxyalkylenated chain and/or at least one glycerylated chain, and more preferably silicone elastomer comprising at least one oxyethylenated chain.

The amount of the (e) emulsifying silicone elastomer(s) in the composition according to the present invention may be 20% by weight or less, preferably 15% by weight or less, and more preferably 10% by weight or less, relative to the total weight of the composition.

The amount of the (e) emulsifying silicone elastomer(s) in the composition according to the present invention may be 0.1% by weight or more, preferably 0.5% by weight or more, and more preferably from 1% by weight or more, relative to the total weight of the composition.

Thus, the amount of the (e) emulsifying silicone elastomer(s) in the composition according to the present invention may range from 0.1% to 20% by weight, preferably from 0.5% to 15% by weight, and more preferably from 1% to 10% by weight, relative to the total weight of the composition.

### Emulsifier

The composition according to the present invention may comprise at least one (f) emulsifier different from the (f) emulsifying silicone elastomer. If two or more emulsifiers are used, they may be the same or different.

The types of the (f) emulsifier are not limited. Thus, for example, amphiphilic powder(s) may be used as the (f) emulsifier. In that case, the composition according to the present invention may be in the form of a Pickering emulsion.

It is preferable that the (f) emulsifier be selected from surfactants.

Thus, the composition according to the present invention may include at least one surfactant. Two or more surfactants may be used in combination. Thus, a single type of surfactant or a combination of different types of surfactant may be used.

The surfactant used in the present invention may be selected from the group consisting of anionic surfactants, amphoteric surfactants, cationic surfactants, and nonionic surfactants, preferably from nonionic surfactants.

It is more preferable that the (f) emulsifier be selected from nonionic silicone surfactants.

It is even more preferable that the (f) emulsifier be selected from nonionic non-crosslinked silicone surfactants. The nonionic non-crosslinked silicone surfactants may be based on linear organopolysiloxanes.

Examples of nonionic silicone surfactants that may be mentioned include dimethicone copolyols, such as the mixture of cyclomethicone and dimethicone copolyol sold under the trade name DC 5225 C by the company Dow Corning, and alkyl dimethicone copolyols such as the lauryl dimethicone copolyol sold under the name Dow Corning 5200 Formulation Aid by the company Dow Corning, and the cetyl dimethicone copolyol sold under the name Abil EM 90™ by the company Goldschmidt.

The nonionic silicone surfactants may be selected from the group consisting of cyclotetrasiloxane (and) cyclopentasiloxane (and) PEG/PPG-18 dimethicone, cyclopentasiloxane (and) PEG/PPG-18/18 dimethicone; PEG/PPG-18/18 dimethicone; lauryl PEG/PPG-18/18 methicone; cetyl PEG/PPG-14/14 dimethicone; bis-cetyl PEG/PPG-14/14 dimethicone; cetyl PEG/PPG-10/1 dimethicone; PEG-11 methyl ether dimethicone; PEG/PPG-20/22 butyl ether dimethicone; PEG-9 dimethicone; PEG-3 dimethicone; PEG-9 methyl ether dimethicone; PEG-10 dimethicone; lauryl PEG-9 polydimethylsiloxyethyl dimethicone.

Among nonionic silicone surfactants, mention may be made of, as usable oxyalkylenated polyorganosiloxane emulsifier, the following:

An oxyalkylenated polyorganosiloxane emulsifier having the general formula:

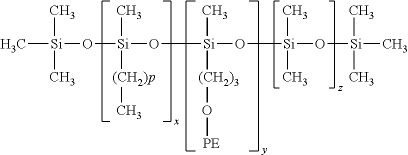

wherein p is 0-40 (the range including all numbers there between and subranges thereof such as 2, 3, 4, 13, 14, 15, 16, 17, 18, etc.), and PE is (—C₂H₄O—)ₐ—(—C₃H₆O—)_{b}—H wherein a is 0-25, b is 0-25 with the proviso that both a and b cannot be 0 simultaneously, x, y, and z are each independently ranging from 0 to 1 million with the proviso that x and y cannot be 0 simultaneously. In some cases, x, y, z, a, and b are such that the molecular weight of the polymer ranges from about 5,000 to about 500,000, from about 10,000 to 100,000, or is about 50,000, and the polymer is generically referred to as dimethicone copolyol. In some instances, p is such that the long chain alkyl is cetyl or lauryl, and the compound is called, generically, cetyl dimethicone copolyol or lauryl dimethicone copolyol, respectively. In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or ether, such as methyl ether, ethyl ether, propyl ether, butyl ether, and the like.

An oxyalkylenated polyorganosiloxane emulsifier having the general formula:

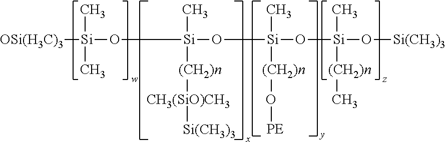

wherein each n is independently 0-100 with the proviso that there must be at least one PE radical. In some instances, where each n independently ranges from about 2 to 30, and PE (—C₂H₄O—)ₐ—(—C₃H₆O—)_{b}—H wherein a is 0-25, b is 0-25 with the proviso that both a and b cannot simultaneously be 0; and wherein w, x, y, and z are each independently 0 to 1,000,000 with the proviso that there is at least one PE. In some embodiments the organosiloxane emulsifier is lauryl PEG-9 Polydimethylsiloxyethyl Dimethicone. Oxyalkylenated organosiloxane emulsifiers disclosed in U.S. Pat. No. 9,095,543 are useful in the instant compositions. U.S. Pat. No. 9,095,543 is incorporated herein by reference in its entirety.

Further examples of polyorganosiloxane emulsifiers as nonionic silicone surfactants include those having the C.T.F.A. names Bis-Butyldimethicone Polyglyceryl-3; Bis-PEG/PPG-14/14 Dimethicone; Bis-butyldimethicone Polyglyceryl-3; Bis-isobutyl PEG/PPG-10/7 Dimethicone copolymer; Bis-PEG/PPG-18/6 Dimethicone; Bis-PEG/PPG-20/20 Dimethicone; Bis-PEG/PPG-16/16 PEG/PPG-16/16 Dimethicone; Bis(PPG-7 Undeceneth-21-Dimethicone; Cetyl Dimethicone PEG-7 Acetate; Cetyl PEG-8 Dimethicone; Cetyl PEG/PPG-15/16 Butyl Ether Dimethicone; Cetyl PEG/PPG-15/15 Butyl Ether Dimethicone; Cetyl PEG/PPG-7/3 Dimethicone; Cetyl PEG/PPG-10/1 Dimethicone; Dimethicone PEG-15 Acetate; Dimethicone PEG-7 Cocoate; Dimethicone PEG-7 Phosphate; Dimethicone PEG-10 Phosphate; Dimethicone PEG/PPG-7/4 Phosphate; Dimethicone PEG/PPG-12/4 Phosphate; Dimethicone PEG-7 Undecylenate; Lauryl Dimethicone PEG-10 Phosphate; Isopolyglyceryl-3 Dimethicone; Isopolyglyceryl-3 Dimethiconol; Isostearyl Carboxyldecyl PEG-8 Dimethicone; Lauryl Methicone PEG-10 Phosphate; Lauryl PEG-8 Dimethicone; Lauryl PEG-10 Methyl Ether Dimethicone; Lauryl PEG/PPG-18/18 Methicone; PEG-6 Methyl Ether Dimethicone; PEG-7 Methyl Ether Dimethicone; PEG-9 Methyl Ether Dimethicone; PEG-10 Methyl Ether Dimethicone; PEG-11 Methyl Ether Dimethicone; PEG-11 Methyl Ether Dimethicone; PEG-32 Methyl Ether Dimethicone; PEG-PEG/PPG-28/21 Acetate Dimethicone; PEG/PPG-22/22 Butyl Ether Dimethicone; PEG/PPG-23/23 Butyl Ether Dimethicone; PEG/PPG-24/18 Butyl Ether Dimethicone; PEG/PPG-3/10 Dimethicone; PEG/PPG-4/12 Dimethicone; PEG/PPG-6/11 Dimethicone; PEG/PPG-8/14 Dimethicone; PEG/PPG-12/16 Dimethicone; PEG/PPG-12/18 Dimethicone; PEG/PPG-14/4 Dimethicone; PEG/PPG-15/5 Dimethicone; PEG/PPG-15/15 Dimethicone; PEG/PPG-16/2 Dimethicone; PEG/PPG-16/8 Dimethicone; PEG/PPG-17/18 Dimethicone; PEG/PPG-18/12 Dimethicone; PEG/PPG-19/19 Dimethicone; PEG/PPG-20/6 Dimethicone; PEG/PPG-20/15 Dimethicone; PEG/PPG-20/20 Dimethicone; PEG/PPG-20/29 Dimethicone; PEG/PPG-22/23 Dimethicone; PEG/PPG-22/24 Dimethicone; PEG/PPG-25/25 Dimethicone; PEG/PPG-27/27 Dimethicone; PEG/PPG-30/10 Dimethicone; PEG/PPG-10/3 Oleyl Ether Dimethicone; PEG-8 trisiloxane; Polyglyceryl-3 Polydimethylsiloxyethyl Dimethicone; PPG-12 Butyl Ether Dimethicone; Silicone Quaternium-17; TEA-Dimethicone PEG-7 Phosphate; or mixtures thereof.

Further examples of commercial linear polyorganosiloxane emulsifiers as nonionic silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Coming 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

It is preferable that the (e) emulsifier be selected from polyoxyalkylene-polyorganosiloxane copolymers, and more preferably selected from the group consisting of PEG 10 dimethicone, PEG/PPG-18/18 dimethicone, and a mixture thereof.

The amount of the (e) emulsifier(s) in the composition according to the present invention may be 30% by weight or less, preferably 25% by weight or less, and more preferably 20% by weight or less, relative to the total weight of the composition.

The amount of the (e) emulsifier(s) in the composition according to the present invention may be 0.001% by weight or more, preferably 0.005% by weight or more, and more preferably from 0.01% by weight or more, relative to the total weight of the composition.

Thus, the amount of the (e) emulsifier(s) in the composition according to the present invention may range from 0.001% to 30% by weight, preferably from 0.005% to 25% by weight, and more preferably from 0.01% to 20% by weight, relative to the total weight of the composition.

### Other Optional Additives

The composition according to the present invention may also comprise any other optional additive(s) usually used in the field of cosmetics, chosen, for example, from anionic, cationic, amphoteric or nonionic polymers; solvents, hydrophilic thickening agents, dispersants, antioxidants, film-forming agents, preserving agents, fragrances, neutralizers, pH adjusting agents, antiseptics, UV-screening agents, cosmetic active agents other than ingredient (a), such as vitamins, moisturizers, emollients or collagen-protecting agents, and mixtures thereof.

It is a matter of routine operations for a person skilled in the art to adjust the nature and amount of the above optional additives which may be present in the composition in accordance with the present invention such that the desired cosmetic properties are not thereby affected.

As the solvents, mention may be made of one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ether, and butylene glycol monomethyl, monoethyl, and monobutyl ethers.

The organic solvent(s) may be present in a concentration of from 0.01% to 35% by weight, preferably from 0.1% to 30% by weight, and more preferably from 1% to 25% by weight, relative to the total weight of the composition.

As the pH adjusting agent, at least one acidifying agent and/or at least one basifying agent (alkaline agent) may be used.

The acidifying agents can be, for example, mineral or organic acids, for instance hydrochloric acid, phosphoric acid, carboxylic acids, for instance tartaric acid, citric acid, and lactic acid, or sulphonic acids.

The basifying agent or alkaline agent can be, for example, any inorganic or organic basic agents which are commonly used in cosmetic products such as ammonia; alkanolamines such as mono-, di- and tri-ethanolamine, isopropanolamine; metal hydroxide such as alkaline metal hydroxide (e.g., sodium and potassium hydroxides); urea, guanidine and their derivatives; and diamines such as those described in the structure below:

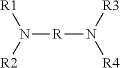

wherein

R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine, and derivatives thereof. Alkaline metal hydroxide such as sodium hydroxide or potassium hydroxide may be preferable.

The acidifying agent and/or at least one basifying agent may be present in an amount ranging from less than 5% by weight, preferably from 3% by weight or less, and more preferably from 1% by weight or less, relative to the total weight of the composition.

### Preparation

The composition according to the present invention can be prepared by mixing the above-described essential and optional ingredients in a conventional manner.

For example, the composition according to the present invention can be prepared by a process comprising the steps of
mixing
an oil phase comprising the (b) at least one oil,
an aqueous phase comprising the (a) thiopyridinone compound and (c) water to disperse the aqueous phase in the oil phase,
such that the weight ratio of the aqueous phase to the oil phase is 1.0 to 10.0.

It is possible to further mix any of the optional ingredients.

The mixing can be performed at any temperature such as room temperature (e.g., 20-25° C., preferably at 25° C.), preferably at a temperature of 30° C. or more, preferably 40° C. or more, and more preferably 50° C. or more. It is preferable to further mix with any of the above-described optional ingredients such as a pH adjusting agent.

The form of the composition according to the present invention is a W/O type in which the oil phase forms a continuous phase, and the aqueous phase forms a discontinuous phase. It is preferable that the composition according to the present invention be in the form of an emulsion or gel, preferably a W/O emulsion or a W/O gel, and more preferably a W/O gel emulsion.

### Cosmetic Process

The composition according to the present invention may be used as a cosmetic or dermatologic composition, preferably a cosmetic composition, and more preferably a cosmetic composition for a keratin substance. As the keratin substance, mention may be made of the skin, scalp, hair, mucosa such as lips, and nails.

The composition according to the present invention may be used as a depigmenting, bleaching or whitening product for a keratinous substance such as skin. In particular, the composition according to the present invention may be used as a whitening product.

The composition according to the present invention may preferably be intended for application onto a keratin substance such as the skin, scalp and/or the lips, preferably the skin.

Thus, the composition according to the present invention can be used for a cosmetic process for a keratin substance, preferably the skin. In one embodiment, the present invention relates to a cosmetic process, preferably a whitening process, for a keratin substance, preferably skin, comprising the step of applying onto the keratin substance the composition according to the present invention.

The composition according to the present invention can be used as a topical cosmetic composition in the form of a lotion, a milky lotion, a cream, a gel, a paste, a serum, foam, or spray.

### Use

The present invention also relates to a use of a W/O composition comprising
a continuous oil phase comprising (b) at least one oil, and
a discontinuous aqueous phase comprising (c) water, dispersed in the oil phase, wherein the weight ratio of the aqueous phase to the oil phase is 1.0 to 10.0,
in order to stabilize (a) at least one compound of formula (I)

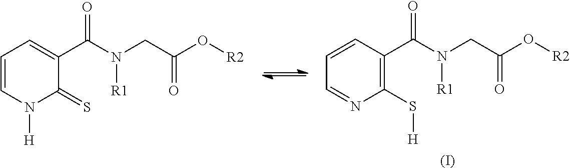

wherein

R1 denotes a radical chosen from:
a) a hydrogen atom; and
b) a saturated linear C₁-C₆ alkyl group,
   and

R2 denotes a radical chosen from:
a) a hydrogen atom;
b) a saturated linear C₁-C₁₀ alkyl group;
c) a saturated branched C₃-C₁₀ alkyl group; and
d) a C₁-C₆ phenylalkyl group such as benzyl,
   or
   a salt thereof, a solvate thereof, an optical isomer thereof, or a racemate thereof, wherein
   the (a) at least one compound of formula (I) is present in the aqueous phase of the composition.

The term “stabilize” has the same meaning as enhancing stability.

The use according to the present invention can increase the stability of the (a) thiopyridinone compound in the composition comprising the same.

Therefore, the use according to the present invention can make it possible to store a composition comprising the (a) thiopyridinone compound for a long period of time under both ambient and hot conditions, and in particular even under hot conditions.

The above explanations regarding the (a) thiopyridinone compound, the (b) oil and the (c) water for the compositions according to the present invention can also apply to those used in the use according to the present invention.

The composition used in the use according to the composition may include any of the optional ingredients as explained above for the compositions according to the present invention.

### EXAMPLES

The present invention will be described in a more detailed manner by way of examples. However, these examples should not be construed as limiting the scope of the present invention.

### Examples 1 and 2

### Preparation

Each of the compositions according to Examples 1 and 2 was prepared by mixing the ingredients shown in Table 1 in accordance with the following Steps 1-6.
1. The ingredients for Phase A1 were mixed and heated to 50° C.+/−5° C. to obtain a mixture of Phase A1 as a clear solution, followed by cooling to room temperature.
2. The ingredients for Phase A2 were added to the mixture of Phase A1 and mixed to obtain a mixture of Phase A1 and Phase A2 as a clear solution.
3. The ingredients for Phase B2 were mixed and heated to 80° C.+/−5° C. to obtain a mixture of Phase B2 as a clear liquid, followed by cooling to room temperature.
4. The ingredients for Phase B1 were added to the mixture of Phase B2 and mixed to obtain a mixture of Phase B1 and Phase B2 as a homogeneous liquid.
5. The mixture of Phase A1 and Phase A2 was added to the mixture of Phase B1 and Phase B2, and the mixture thus obtained was homogenized with a homogenizer (TK robomix, PRIMIX) at 8,000 rpm at room temperature for 10 minutes.
6. The ingredient for Phase C was further added to the above mixture obtained in Step 5, followed by homogenizing with the homogenizer at 8,000 rpm at room temperature for 10 minutes.

The numerical values for the amounts of the ingredients are all based on “% by weight” as raw materials.

TABLE 1

| Phase | Ingredients | Ex. 1 | Ex. 2 |
|---|---|---|---|
| A1 | Methylparaben | 0.20 | 0.20 |
| A1 | Propylene Glycol | 6.00 | 6.00 |
| A1 | Water | 32.89 | 32.13 |
| A1 | Disodium EDTA | 0.05 | 0.05 |
| A1 | Glycerin | 23.00 | 23.00 |
| A2 | Thiopyridinone | 0.50 | 1.00 |
| A2 | Potassium Hydroxide | 0.26 | 0.52 |
| B1 | PEG/PPG-18/18 Dimethicone 12 wt % | 20.00 | 20.00 |
| | Cyclopentasiloxane 86.4 wt % | | |
| | Water 1.6 wt % | | |
| B1 | Phenyltrimethicone | 4.00 | 4.00 |
| B1 | Dimethicone (and) Dimethicone/Vinyl | 5.00 | 5.00 |
| | Dimethicone Crosspolymer | | |
| B2 | Prunus Armeniaca (Apricot) Kernel Oil | 3.00 | 3.00 |
| B2 | Propylparaben | 0.10 | 0.10 |
| C | Nylon12 | 5.00 | 5.00 |
| | | 100 | 100 |

### Comparative Examples 1 and 2

### Preparation

Each of the compositions according to Comparative Examples 1 and 2 was prepared by mixing the ingredients shown in Table 2 in accordance with the following Steps 1-6.
1. The ingredients for Phase A were mixed and heated to 80° C.+/−5° C. to obtain a mixture of Phase A as a clear solution.
2. The ingredients for Phase B were mixed and heated to 80° C.+/−5° C. to obtain a mixture of Phase B.
3. The mixture of Phase B was added to the mixture of Phase A, and the mixture thus obtained was homogenized with a homogenizer (TK robomix, PRIMIX) at 8,000 rpm at 80° C.+/−5° C.
4. The ingredient for Phase C was further added to the above mixture obtained in Step 3, followed by homogenizing with the homogenizer at 8,000 rpm at spontaneous temperature for 10 minutes.
5. The ingredients for Phase D were mixed by stirring to obtain a mixture of Phase D.
6. The mixture of Phase D was added to the above mixture obtained in Step 4, followed by homogenizing with the homogenizer at 8,000 rpm at room temperature for 5 minutes.

The numerical values for the amounts of the ingredients are all based on “% by weight” as raw materials.

TABLE 2

| | | Comp. | Comp. |
|---|---|---|---|
| Phase | Ingredients | Ex. 1 | Ex. 2 |
| A | Pentylene Glycol | 3.00 | 3.00 |
| A | Water | 57.7 | 56.88 |
| A | Sodium Stearoyl Glutamate | 0.25 | 0.25 |
| A | Glycerin | 3.00 | 3.00 |
| A | Propanediol | 5.00 | 5.00 |
| A | Caprylyl Glycol | 0.30 | 0.30 |
| A | Phenoxyethanol | 0.50 | 0.50 |
| B | Sucrose Tristearate | 2.00 | 2.00 |
| B | Limnanthes Alba (Meadowfoam) seed oil | 5.00 | 5.00 |
| B | Behenyl Alcohol | 2.00 | 2.00 |
| C | Xanthan Gum | 0.20 | 0.20 |
| C | Acrylates/C10-30 Alkyl Acrylate Crosspolymer | 0.20 | 0.20 |
| D | Water | 20.00 | 20.00 |
| D | Thiopyridinone | 0.50 | 1.00 |
| D | Potassium Hydroxide | 0.35 | 0.67 |
| | | 100 | 100 |

### Evaluations

### Thiopyridinone Quantification

The amount of thiopyridinone in each of the compositions according to Examples 1 and 2 and Comparative Examples 1 and 2 was determined by an HPLC-UV assay at the following timing.
(1) Just after the preparation of the composition (T0)
(2) Two months after the preparation, where the composition was maintained at room temperature
(3) Two months after the preparation, where the composition was maintained at 45° C.

The details of the HPLC-UV assay are as follows.

### Apparatus/Reagents

| | |
|---|---|
| HPLC System | HPLC with UV detector |
| HPLC Column | C8 column 5 μm, 4 mm × 250 mm |
| Acetonitrile | HPLC grade |
| Monopotassium phosphate | Special grade |

### HPLC Conditions

| | |
|---|---|
| UV detector | 220 nm |
| Column Temp. | 30° C. |
| Flow Rate | 1.0 mL/min |
| Injection Volume | 5 μL |
| Mobile Phase | Gradient mode |
| | A: 0.01 mol/L monopotassium phosphate solution, |
| | pH = 3.0 with phosphoric acid |
| | B: Acetonitrile |

The results are shown in Table 3.

### Texture Evaluation

Each of the compositions according to Examples 1 and 2 and Comparative Examples 1 and 2 just after the preparation thereof and two months after the preparation was subjected to sensory evaluation by 8 panelists. 0.08-0.12 g of each composition was applied onto the skin of each panelist and evaluated in teams of stickiness. The panelists evaluated in accordance with the following score criteria, and the scores were averaged.

Very Good: more than 6 panelists perceived “not sticky”

Good: more than 4 panelists perceived “not sticky”

Fair: more than 2 panelists perceived “not sticky”

Poor: equal to or less than 2 panelists perceived “not sticky”

The results are shown in the “Stickiness” line in Table 3.

TABLE 3

| | | | Comp. | Comp. |
|---|---|---|---|---|
| | Ex. 1 | Ex. 2 | Ex. 1 | Ex. 2 |
| Structure | W/O | W/O | O/W | O/W |
| Thiopyridinone | 0.50 | 1.00 | 0.50 | 1.00 |
| Theoretical Amount (wt %) | | | | |
| Thiopyridinone | 0.49 | 1.03 | 0.50 | 1.11 |
| T0 Amount (%) | | | | |
| Thiopyridinone | 0.49 | 0.993 | 0.49 | 1.01 |
| RT 2 M Amount (%) | | | | |
| Thiopyridinone | 0.46 | 0.933 | 0.41 | 0.925 |
| 45° C. 2 M Amount (%) | | | | |
| 45° C. 2 M/RT 2 M (%) | 93.9 | 93.9 | 83.7 | 91.1 |
| No Stickiness | Very good | Very good | Fair | Poor |

Thiopyridinone Theoretical Amount (wt %): Amount of thiopyridinone used in the preparation of the composition.

Thiopyridinone T0 Amount (%): Amount of thiopyridinone just after the preparation of the composition (determined by HPLC)

Thiopyridinone RT 2M Amount (%): Amount of thiopyridinone two months after the preparation of the composition, where the composition was maintained at room temperature (determined by HPLC)

Thiopyridinone 45° C. 2M Amount (%): Amount of thiopyridinone two months after the preparation of the composition, where the composition was maintained at 45° C. (determined by HPLC)

45° C. 2M/RT 2M (%): {Amount of thiopyridinone two months after the preparation of the composition at 45° C./Amount of thiopyridinone two months after the preparation of the composition at room temperature}*100

Table 3 shows that, in cases wherein thiopyridinone was included in the compositions according to Comparative Examples 1 and 2 in the form of typical O/W emulsions, the amount of thiopyridinone decreased more than 10% when the compositions were stored at 45° C. for two months, compared with room temperature for two months.

On the other hand, Table 3 also shows that, in cases wherein thiopyridinone was included in the compositions according to Examples 1 and 2 in the form of W/O emulsions, the reduction in amount of thiopyridinone was less than 10% when the compositions were stored at 45° C. for two months, compared with room temperature for two months.

In addition, the stickiness of the compositions according to Examples 1 and 2 was better than that of the compositions according to Comparative Examples 1 and 2, respectively.

### Example 3

### Preparation

The composition according to Example 3 was prepared by mixing the ingredients shown in Table 4 in accordance with the following Steps 1-5.
1. The ingredients for Phases A1 and A2 were mixed at room temperature to prepare a homogeneous solution
2. The ingredients for Phase B1 were mixed and heated to 50° C.+/−5° C. to obtain a clear solution, followed by cooling to room temperature.
3. The ingredients for Phases B2 and B3 to the clear solution obtained by Step 2 and mixed to obtain a mixture of Phase B1-B3 as a clear solution.
4. The mixture of Phases B1-B3 was added to the mixture of Phases A1 and A2, and the mixture thus obtained was homogenized with a homogenizer (TK robomix, PRIMIX) for 5 minutes, followed by gently mixing with a paddle mixer (Heidon, BL600).
5. The ingredient for Phase C was further added to the above mixture obtained in Step 4, followed by mixing with the paddle mixer for 10 minutes.

The numerical values for the amounts of the ingredients are all based on “% by weight” as raw materials.

TABLE 4

| Phase | Ingredients | Ex. 3 |
|---|---|---|
| A1 | Dimethicone (and) Dimethicone/PEG-10/15 | 5.00 |
| | Crosspolymer | |
| A1 | PEG-10 Dimethicone | 0.07 |
| A2 | Dimethicone | 7.00 |
| B1 | Water | 20.00 |
| B1 | p-Anisic Acid | 0.15 |
| B1 | Sodium Hydroxide | 0.05 |
| B2 | Water | 43.63 |
| B2 | Glycerin | 15.00 |
| B2 | Propanediol | 5.00 |
| B2 | Phenoxyethanol | 0.50 |
| B2 | Chlorphenesin | 0.27 |
| B2 | Disodium EDTA | 0.10 |
| B2 | Sodium Citrate | 0.20 |
| B2 | Sodium Chloride | 0.50 |
| B2 | Thiopyridinone | 1.00 |
| B2 | Potassium Hydroxide | 0.50 |
| B3 | Citric Acid | 0.03 |
| C | Polymethylsilsesquioxane | 1.00 |
| | | 100 |

### Evaluation

### Thiopyridinone Quantification

The amount of thiopyridinone in the composition according to Example 3 was determined by an HPLC-UV assay at the following timing.
(1) Just after the preparation of the composition (T0)
(2) Two months after the preparation, where the composition was maintained at room temperature
(3) Two months after the preparation, where the composition was maintained at 45° C.

The details of the HPLC-UV assay are the same as those for the thiopyridinone quantification for Examples 1 and 2 described above.

The results are shown in Table 5.

TABLE 5

| | Ex. 3 |
|---|---|
| Structure | W/O |
| Thiopyridinone | 1.00 |
| Theoretical Amount (wt %) | |
| Thiopyridinone | 0.99 |
| T0 Amount (%) | |
| Thiopyridinone | 0.99 |
| RT 2 M Amount (%) | |
| Thiopyridinone | 0.97 |
| 45° C. 2 M Amount (%) | |
| 45° C. 2 M/RT 2 M (%) | 98.0 |
| No stickiness | Very good |

Thiopyridinone Theoretical Amount (wt %): Amount of thiopyridinone used in the preparation of the composition.

Thiopyridinone T0 Amount (%): Amount of thiopyridinone just after the preparation of the composition (determined by HPLC)

Thiopyridinone RT 2M Amount (%): Amount of thiopyridinone two months after the preparation of the composition, where the composition was maintained at room temperature (determined by HPLC)

Thiopyridinone 45° C. 2M Amount (%): Amount of thiopyridinone two months after the preparation of the composition, where the composition was maintained at 45° C. (determined by HPLC)

45° C. 2M/RT 2M (%): {Amount of thiopyridinone two months after the preparation of the composition at 45° C./Amount of thiopyridinone two months after the preparation of the composition at room temperature}*100

Table 5 shows that, in a case wherein thiopyridinone was included in the composition according to Example 3 in the forth of a W/O emulsion, the reduction in amount of thiopyridinone was suppressed within 3% when the composition was stored at 45° C. for two months, compared with room temperature for two months.

1. A W/O composition comprising:
(a) at least one compound of formula (I) 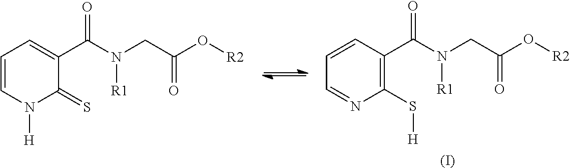
wherein
R1 denotes a radical chosen from:
a) a hydrogen atom; and
b) a saturated linear C₁-C₆ alkyl group,
and
R2 denotes a radical chosen from:
a) a hydrogen atom;
b) a saturated linear C₁-C₁₀ alkyl group;
c) a saturated branched C₃-C₁₀ alkyl group; and
d) a C₁-C₆ phenylalkyl group,
or
a salt thereof, a solvate thereof, an optical isomer thereof, or a racemate thereof;
(b) at least one oil; and
(c) water,
wherein
the composition comprises
a continuous oil phase comprising the (b) at least one oil,
a discontinuous aqueous phase comprising the (a) at least one compound of formula (I) and (c) water, dispersed in the oil phase, and
the weight ratio of the aqueous phase to the oil phase is 1.0 to 10.0.

2. The composition according to claim 1, wherein:
R1 denotes a radical chosen from:
a) a hydrogen atom; and
b) a saturated linear C₁-C₄ alkyl radical,
and
R2 denotes a radical chosen from:
a) a hydrogen atom;
b) a saturated linear C₁-C₆ alkyl group; and
c) a saturated branched C₃-C₆ alkyl group.

3. The composition according to claim 1, wherein:
R1 denotes a radical chosen from:
a) a hydrogen atom; and
b) a methyl radical,
and
R2 denotes a radical chosen from:
a) a hydrogen atom;
b) a saturated linear C₁-C₄ alkyl group;
c) a saturated branched C₃-C₄ alkyl group.

4. The composition according to claim 1, wherein the (a) compound of formula (I) is chosen from the following compounds:

| | Compound | |
|---|---|---|
| Structure | No. | Chemical Name |
| 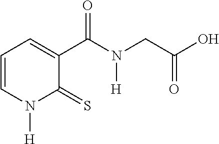 [SMILES: O=C(O)CNC(=O)c1ccc[nH]c1=S] | 1 | N-[(2-thioxo-1,2-dihydro- pyridin-3-yl)carbonyl]- glycine |
| 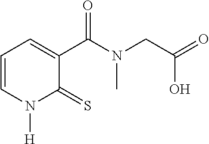 | 2 | N-methyl-N-[(2-thioxo- 1,2-dihydropyridin-3-yl)- carbonyl]glycine |
| 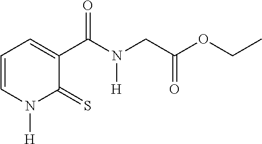 [SMILES: CCOC(=O)CNC(=O)c1ccc[nH]c1=S] | 3 | Ethyl N-[(2-thioxo-1,2- dihydropyridin-3-yl)- carbonyl]glycinate |
| 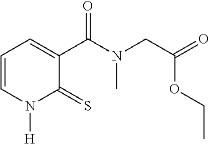 [SMILES: CCOC(=O)CN(C)C(=O)c1ccc[nH]c1=S] | 4 | Ethyl N-methyl-N-[(2- thioxo-1,2-dihydropyridin- 3-yl)carbonyl]glycinate |

and salts thereof, solvates thereof, optical isomers thereof, and racemates thereof.

5. The composition according to claim 1, wherein the (a) compound of formula (I) is chosen from the following compounds:

| | Compound | |
|---|---|---|
| Structure | No. | Chemical Name |
| 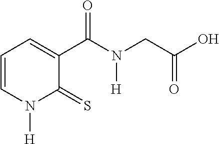 [SMILES: O=C(O)CNC(=O)c1ccc[nH]c1=S] | 1 | N-[(2-thioxo-1,2-dihydropyridin- 3-yl)carbonyl]glycine |
| 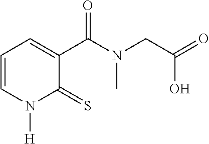 | 2 | N-methyl-N-[(2-thioxo-1,2- dihydropyridin-3-yl)carbonyl]- glycine |

and salts thereof, solvates thereof, optical isomers thereof, and racemates thereof.

6. The composition according to claim 1, wherein the amount of the (a) compound(s) of formula (I) in the composition is from 0.01% to 20% by weight, relative to the total weight of the composition.

7. The composition according to claim 1, wherein the (b) oil is selected from silicone oils.

8. The composition according to claim 1, wherein the amount of the (b) oil(s) in the composition is from 8% to 40% by weight, relative to the total weight of the composition.

9. The composition according to claim 1, wherein the amount of the (c) water in the composition is from 10% to 80% by weight, relative to the total weight of the composition.

10. The composition according to claim 1, wherein the composition further comprises (d) at least one lipophilic gelling agent.

11. The composition according to claim 1, wherein the composition further comprises (e) at least one emulsifying silicone elastomer.

12. The composition according to claim 11, wherein the composition further comprises (f) at least one emulsifier different from the (e) emulsifying silicone elastomer.

13. The composition according to claim 1, wherein the composition is for whitening a keratin substance.

14. A cosmetic process for a keratin substance, comprising:
applying to the keratin substance the composition according to claim 1.

15. A method for stabilizing (a) at least one compound of formula (I) in a W/O composition, the composition comprising:
a continuous oil phase comprising (b) at least one oil, and
a discontinuous aqueous phase comprising (c) water, dispersed in the oil phase, wherein the weight ratio of the aqueous phase to the oil phase is 1.0 to 10.0,
the formula (I) being 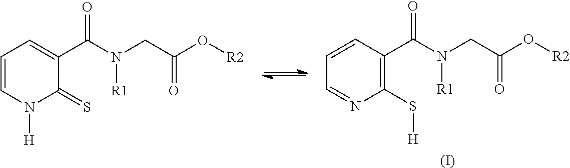
wherein
R1 denotes a radical chosen from:
a) a hydrogen atom; and
b) a saturated linear C₁-C₆ alkyl group,
and
R2 denotes a radical chosen from:
a) a hydrogen atom;
b) a saturated linear C₁-C₁₀ alkyl group;
c) a saturated branched C₃-C₁₀ alkyl group; and
d) a C₁-C₆ phenylalkyl group,
or
a salt thereof, a solvate thereof, an optical isomer thereof, or a racemate thereof,
wherein
the method comprises:
including the (a) at least one compound of formula (I) in the aqueous phase of the composition.
